(19) United States
(12) Patent Application Publication
(10) Pub. No.: US 2003/0022175 A1
(43) Pub. Date: Jan. 30, 2003

(54) Diagnostic assay of genetic mutations by discriminating amplification and hybridization

(76) Inventors: Harn-Jing Terng, Hsinchu (TW); Pei-Hua Wu, Hsinchu (TW); Shin-Hwan Wang, Hsinchu (TW)

Correspondence Address: Y. ROCKY TSAO, Fish & Richardson P.C., 225 Franklin Street, Boston, MA, 02110-2804, US

(21) Appl. No.: 09/915,780

(22) Filed: Jul. 26, 2001

(51) Int. Cl. C12Q 1/68; C07H 21/04; C12P 19/34; C07K 9/00

(52) U.S. Cl. 435/6; 435/91.2; 536/24.3; 530/322

(57) ABSTRACT

This invention features a discrimination primer for amplifying a nucleic acid that includes a first base at a position suspected of a polymorphism and a second base immediately 3′ to the first base. This primer includes (1) a first nucleotide, which is located at the 3′ terminus of the primer and includes a base that is complementary to the first base; (2) a second nucleotide, which is located immediately 5′ to the first nucleotide and includes a base that is not complementary to the second base; (3) a segment of nucleotides, which is located immediately 5′ to the second nucleotide and is complementary to a part of the nucleic acid that is immediately 3′ to the second base; and (4) a binding member of a specific binding pair covalently bonded to the 5′ terminus of the segment. The first base of the nucleic acid can be mutant or wild-type.

Single nucleotide polymorphisms, a set of single nucleotide variants at genomic loci, are distributed throughout a genome. In the human genome, such single nucleotide variation occurs relatively frequently, about once in every 200-1000 bases, resulting in millions of single nucleotide polymorphisms (Collins, et al. (1997) Science 278: 1580). In general, when a single nucleotide polymorphism exists at a locus within a gene for a structure protein, the variant may be dominant. On the other hand, when a single nucleotide polymorphism is at a locus within a gene for a catalytic enzyme, the variant may be recessive. See Beaudet et al. (1989) The Metabolic Basis of Inherited Disease 6^{th} Ed. Scriver et al. (Eds) McGraw-Hill Publishing Co. New York, pp 13. In animals, genetic recessive disorders caused by a polymorphism may include bovine leukocyte adhesion deficiency (BLAD, Shuster et al. (1992) Pro. Acad. Natl. Sci. USA 89: 9225-9229), citrullinemia (Dennis et al. (1989) Pro. Acad. Natl. Sci. USA 86: 7947-7951), maple syrup urine disease (MSUD, Zhang et al. (1990) J. Biol. Chem. 265: 2425), deficiency of uridine monophosphate synthase (DUMPS, Shanks et al. (1987) J. Anim. Sci. 64: 695-700), α-mannosidosis (Jolly (1993) Vet. Clin. N. Am. 9: 41), and generalized glycogenosis (Pompes Disease; Dennis et al. (2000) Mamm. Genome 11: 206). In humans, an example of genetic recessive disorders is cystic fibrosis (Kerem et al. (1989) Science 245: 1073-1080), which affects about 1/2000 individuals of the entire Caucasian population.

A single nucleotide polymorphism can be “allelic.” That is, due to the existence of the polymorphism, some members of a species may have the unmutated sequence (i.e. the wild-type allele) whereas other members may have a mutated sequence (i.e. the mutant allele). Further, for each polymorphism, there are three possible genotypes: homozygous wild-type alleles, homozygous mutant alleles, and heterozygous alleles. There remains a need for an efficient method for detecting a single nucleotide polymorphism, including genotyping.

### SUMMARY

This invention relates a novel primer that discriminates between two nucleic acids which differ by only one base, and therefore, can be used to detect a single nucleotide polymorphism.

More specifically, one aspect of this invention features a discrimination primer for amplifying a nucleic acid that includes a first base at a position suspected of a polymorphism and a second base immediately 3′ to the first base. This primer includes (1) a first nucleotide, which is located at the 3′ terminus of the primer and contains a base that is complementary to the first base; (2) a second nucleotide, which is located immediately 5′ to the first nucleotide and contains a base that is not complementary to the second base; (3) a segment of nucleotides (e.g., 5 to 50, or 10 to 40 nucleotides in length), which is located immediately 5′ to the second nucleotide and is complementary to a part of the nucleic acid that is immediately 3′ to the second base; and (4) a binding member of a specific binding pair covalently bonded to the 5′ terminus of the segment. The first base of the nucleic acid can be mutant or wild-type.

A nucleic acid targeted to be amplified can be DNA (ss or ds DNA) or RNA, in a purified or unpurified form. It also can be a genomic fragment or a restriction fragment. The term “complementary” refers to a sequence forming a duplex with another sequence when these sequences base pair with one another, perfectly or partly. In a perfect duplex, two sequences are precisely complementary. In a partial duplex, two sequences have at least one, two, or more mismatched base pairs, but are still capable of synthesizing a primer extension product.

A specific binding pair refers to two binding members that specifically bind to one another. It can be a protein-ligand pair (e.g., streptavidin-biotin), a hybridizing nucleic acid pair, a protein-protein pair, an antibody-antigen pair, or a nucleic acid-nucleic acid binding protein pair. For example, a binding member of the specific binding pair is an oligonucleotide that is not complementary to any part of the nucleic acid to be amplified, and the other member of the specific binding pair is also an oligonucleotide; both binding members can be 6 to 50 nucleotides (e.g., 10 to 40 nucleotides) in length. One binding member forms an integral part of the discrimination primer, and thus also of an amplification product extended therefrom. Via the binding member, the amplification product binds to the other binding member, which is immobilized (directly or indirectly) on a solid substrate. Affixation of the amplification product to a solid substrate facilitates its detection.

In another aspect, this invention features a method for detecting a polymorphism in a nucleic acid. The method includes (1) providing a nucleic acid containing a base at a position suspected of a polymorphism; (2) amplifying the nucleic acid with a first binding member-containing discrimination primer (as described above) and another primer (an amplification primer); (3) contacting the amplified nucleic acid with a second binding member capable of binding to the first binding member; and (4) detecting the amplified nucleic acid that binds to the second binding member. Optionally, this method includes amplifying the nucleic acid in the presence of two discrimination primers, one of which includes a mutant base in the nucleic acid sequence, and the other primer includes a wild-type base. The term “amplifying” as used herein refers to the process of producing multiple copies of a desired sequence of the provided nucleic acid or a portion thereof, e.g., 50 to 1000 nucleotides in length.

An amplification primer, together with a discrimination primer, is used to amplify a nucleic acid including a polymorphism. It can include a label at its 5′ terminus. The label can be detected, directly or indirectly, by well-known techniques. Examples of the label include, but are not limited to, a fluorescent molecule (e.g., fluorescein and rhodamine), biotin (which can be detected by an anti-biotin specific antibody or an enzyme-conjugated avidin derivative), a radioactive isotope (e.g., ³²P or ¹²⁵I), a calorimetric reagent, and a chemiluminescent reagent.

Also within the scope of this invention is a kit for detecting a polymorphism. The kit includes a discrimination primer and an amplification primer as described above. Optionally, the kit can include two discrimination primers, one containing a mutant base at its 3′ terminus, and the other containing a wild-type base at its 3′ terminus. When only one discrimination primer is included, the kit can be used to analyze a polymorphism. When two discrimination primers are included, the kit can be used to further determine the genotype of polymorphic alleles. In addition to the primers, the kit may further include an enzyme (i.e., DNA polymerase) and reagents for amplification (e.g., nucleotides, or analogs thereof such as deoxyinosine). It can also include solid substrates, such as glass plates or plastic microchips containing arrays of oligonucleotides (i.e., various binding members to be used to bind amplification products).

Other features, objects, and advantages of the invention will be apparent from the description and from the claims.

### DETAILED DESCRIPTION

The present invention relates to a discrimination primer, and an amplification primer, for use in an amplification reaction to detect a polymorphism.

A “primer” is an oligonucleotide capable of acting as a point of initiation of synthesis of a primer extension product that is complementary to a nucleic acid strand (template or target sequence), when placed under suitable conditions (e.g., salt concentration, temperature, and pH) in the presence of nucleotides and other reagents for nucleic acid polymerization (e.g., a DNA dependent polymerase). As known in the art, a primer must be of a sufficient length (e.g., at least 6 nucleotides) to prime the synthesis of extension products.

Use of a discrimination primer and an amplification primer allows for preferential (e.g., exclusive) amplification of a nucleic acid that contains a polymorphism. In other words, such a primer pair can be used to preferentially amplify one polymorphic allele (e.g., mutant allele) over the other (e.g., wild-type allele). The discrimination primer includes a first nucleotide, which is located at the 3′ terminus of the primer and contains a base that is complementary to a first base suspected of a polymorphism in a nucleic acid; a second nucleotide, which is located immediately 5′ to the first nucleotide and contains a base that is not complementary to a second base immediately 3′ to the first base; a segment of nucleotides; and a binding member of a specific binding pair. The oligonucleotide consisting of the first and second nucleotides and the segment (at least 5 bases in length) is capable of acting as a point of initiation of synthesis of a primer extension product. When the first base in the nucleic acid is wild-type, a discrimination primer containing a mutant nucleotide at its 3′ terminus has one more mismatched base when annealing to a wild-type allele than annealing to a mutant allele. More specifically, this discrimination primer has two mismatched bases at its 3′ end when annealing to a wild-type allele, and is therefore not able to act as a point of initiation of synthesis of a primer extension reaction. Conversely, when the first base is mutant, the just-described discrimination primer has only one mismatched base at its 3′ end, and can be used to act as a point of initiation of synthesis of a primer extension reaction.

A discrimination primer can be optimized on a gene-by-gene basis to provide the greatest degree of discrimination between amplification of the wild-type allele and the mutant allele. The optimization will of necessity include some empirical observations, but a number of basic principles can be applied to select a suitable starting point for final optimization. A discrimination primer can be designed based on a known single nucleotide polymorphism in a gene, and also based on its properties, e.g., GC-content, annealing temperature, or internal pairing, which can be analyzed using software programs. As discussed above, a discrimination primer of this invention includes a first binding member, such that an amplification product can bind, via the first binding member, to a second binding member immobilized on a solid substrate. If both binding members are oligonucleotides, the optimization may further take account of annealing or other properties of the first and second binding members. Of course, one must confirm empirically the ability of a discrimination primer to amplify a mutant allele or a wild-type allele.

A primer pair of this invention can be used to selectively amplify a nucleic acid with a single nucleotide polymorphism. The nucleic acid can be obtained from any suitable source, e.g., a tissue homogenate, blood, amniotic fluid, or chorionic villus samples; and can be DNA or RNA (in the case of RNA, reverse transcription is required before PCR amplification). PCR amplification can be carried out following standard procedures. See, e.g., Ausubel et al. (1989) Current Protocols in Molecular Biology John Wiley and Sons, New York; Innis et al. (1990) PCR Protocols: A Guide to Methods and Applications Academic Press, Harcourt Brace Javanovich, New York. More specifically, a method of discriminating amplification has been described in, for example, Cha et al. (1992) PCR Methods and Applications 2: 14. Unexpectedly, the discrimination primer of this invention, despite the presence of a first binding member at its 5′ terminus, can still efficiently produce a specific amplification product. A discrimination primer that contains an oligonucleotide as the first binding member can be prepared by a synthetic method, or alternatively, by a ligation method (e.g., a method of using cyanogen bromide described in Selvasekaran and Turnbull (1999) Nucleic Acids Res. 27(2): 624). A discrimination primer that contains a peptide as the first binding member can be prepared by conjugation of a peptide and an oligonucleotide based on, e.g., a “native ligation” of an N-terminal thioester-functionalized peptide to a 5′-cysteinyl oligonucleotide. See Stetsenko and Gait (2000) J. Org. Chem. 65(16): 4900.

Detection of an amplification product of a polymorphism-containing nucleic acid indicates the presence of a wild-type or mutant allele. According to the method of this invention, an amplification product is detected on a solid substrate when a first binding member, an integral part of the amplification product, binds to a second binding member that is immobilized on a solid substrate. Affixing the amplification product to a solid substrate facilitates its detection. The second binding member can be directly immobilized on a solid support. It also can be indirectly immobilized on a solid substrate. More specifically, if the second binding member has a segment binds to the first binding member and has another segment that binds to a third binding member that has been immobilized on a solid substrate, it can be immobilized on the solid substrate via binding to the third binding member.

One can immobilize a second binding member on a solid substrate by attaching it to the substrate via a covalent or non-covalent bonding. Alternatively, a second binding member can be formed on the substrate by attaching a precursor molecule to the substrate and subsequently converting the precursor to the second binding member, such as de novo synthesis of nucleic acid at a precise region on the solid substrate by a photolithographic method. For example, see, Schena et al. (1995) Science 270: 467; Kozal et al. (1996) Nature Medicine 2(7): 753; Cheng et al. (1996) Nucleic Acids Res. 24(2): 380; Lipshutz et al. (1995) BioTechniques 19(3): 442; Pease et al. (1994) Proc. Natl. Acad. Sci. USA 91: 5022; Fodor et al. (1993) Nature 364: 555-; and Fodor et al., WO 92/10092. The solid substrate can be an agarose, acrylamide, or polystyrene bead; a nylon or nitrocellulose membrane (for use in, e.g., dot or slot blot assays); a glass or plastic polymer; a silicon or silicon-glass (e.g., a microchip); or gold (e.g., gold plates).

An amplification product is detected after it binds to an immobilized second binding member. To enable detection, the amplification product can be labeled by using a labeled amplification primer, or can be labeled, chemically or enzymatically, after amplification. When only the amplification product or the second binding member is labeled with a fluorescent molecule, the presence of the amplification product can be detected by fluorescence. When both the amplification product and the second binding member are labeled with fluorophores, the amplification product can be detected by monitoring a color shift due to proximity of the fluorophores resulting from binding of the amplification product to the second binding member. Examples of fluorescent labels include, but are not limited to, fluorescein, rhodamines, infrared dyes (e.g., IR-132 or IR-144; Kodak, Rochester, N.Y), and cyanine dyes (e.g., Cy3 or Cy5; Amersham Int'l, Cleveland). See Ranki et al. (1983) Gene 21: 77: Keller et al. (1991) J. Clin. Microbiol. 29: 638; and Urdea et al. (1987) Gene 61: 253.

To determine a genotype at a locus of a polymorphism, an assay can be performed as follows. Two discrimination primers and one amplification primer are used in PCR amplification. One discrimination primer has the 3′ terminal nucleotide complementary to a mutant base, and is for use to preferentially amplify a mutant allele. The other discrimination primer has the 3′ terminal nucleotide complementary to a wild-type base, and is for use to preferentially amplify a wild-type allele. The first binding members of the two discrimination primers are different oligonucleotides, and bind to different binding partners (i.e., second binding members), which are separately immobilized. Binding of an amplification product to one immobilized binding partner, to the other immobilized binding partner, or to both indicates one of the three possible genotypes. Unexpectedly, this assay has a high sensitivity, i.e., up to 100 folds that an assay in which amplification products are detected on agarose gel.

Use of spatially arrayed binding partners for simultaneously detecting a multiplicity of polymorphisms is within the scope of this invention. See, for example, U.S. Pat. Nos. 5,424,186, 5,510,270, and 5,744,305.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

### EXAMPLE 1

### Detection of a Single Nucleotide Polymorphism Using Plasmid DNA

Construction of standard BLAD gene fragments. A cattle recessive genetic disorder, bovine leukocyte adhesion deficiency (BLAD), is caused by a single nucleotide polymorphism (Shuster et al. (1992) Proc. Natl. Acad. Sci. U.S.A. 89: 9225-9229). The study was based on the published gene sequence BTCD18, partial sequence of B. taurus CD18 gene (NCBI, Accession No. Y12672). The single point mutation is located at nucleotide position 1200 of BTCD18 gene.

A 1341 bp fragment was amplified from the BTCD18 gene, incorporated in a pGEM-T Easy vector system (Promega, Madison, Wis., USA), and transformed in a bacterium host, Escherichia coli. Nucleic acids were prepared from all transformants and analyzed using the restriction enzyme TaqI. The presence or absence of a TaqI-restriction site in a nucleic acid indicates the type of an allele. A wild-type allele possesses the TaqI restriction site, while a mutant allele does not. Two transformants with plasmids pGEM7-BD and pGEM8-BD were selected as representative standard gene fragments for wild-type allele and mutant allele, respectively. Their sequences were confirmed by sequence analysis.

Discriminating amplification of two genetic alleles. Discriminating amplification was performed by employing one amplification primer as a forward primer and two discrimination primers as reverse primers. HjT-F8a was designed as an amplification primer. It was 23 nucleotides in length, corresponding to 1020-1042 of the BTCD18 gene sequence: 5′-GAATTCACCAGCATAAGAGAATGGGGAG-3′ (SEQ ID NO:1), and had biotin at its 5′-terminus. Two discrimination primers were R11-1-3mis18 (wild-type allele specific reverse primer), 5′-AGTTCTAGAGCGCTCGAGCCATCAGGTAGTACAGAT-3′ (SEQ ID NO:2), and RM11-1-3mis18 (mutant allele specific reverse primer), 5′-GAGTCGTATTACGGATCCTCCATCAGGTAGTACAGAC-3′ (SEQ ID NO:3). The two primers were based on 1218-1200 and 1217-1200 of the BTCD18 gene sequence, respectively, and their first binding members are underlined. The ultimate 3′ base of wild-type (mutant) specific reverse primer was designed as a complementary base to the wild-type (mutant) allele. Each reverse primer had one mismatched base at penultimate position of the 3′-terminus. All primers were commercially prepared by standard oligonucleotide synthesis techniques (e.g., GENESET Singapore Biotech. Pte Ltd, Singapore).

An amplification reaction was performed as follows. The reaction volume was 50 μL and the reaction mixture contained 50-100 ng DNA template, 5 μL 10×Taq DNA polymerase buffer, 5 μL 15 mM MgCl₂, 250 μM dNTP each (Promega), 400 nM primer each, 2-2.5 units Taq DNA polymerase (Promega), and dH₂O. A two-step amplification reaction was employed with temperature parameters of 94° C. for 30 sec and 55° C. for 20 sec for 30-35 cycles after one cycle with 94° C. for 4 min. Amplification conditions were carried out using RoboCycler Temperature Cycler (Strategene).

The amplification products were analyzed by using agarose gel electrophoresis. The gel showed that a specific amplification product with the right size (222 bp) was produced when the primers HjT-F8a and R11-1-3mis18 were mixed with the template pGEM7-BD (the wild-type allele), but not with the template pGEM8-BD (the mutant allele). Similarly, the primers HjT-F8a and RM11-1-3mis18 were able to amplify the mutant allele pGEM8-BD, but were not able to amplify the wild-type allele pGEM7-BD.

Analysis of the amplification products by hybridization. Three types of the second binding members (i.e., oligonucleotides) were designed for analysis of amplification products of the mutant allele and the wild-type allele. Type 1 oligonucleotides were designed to identify the amplification products, i.e., HjT-P1, 5′-(T)₂₅-CTGATGGAGGATCCGTAATACGACTC-3′ (SEQ ID NO:4), and HjT-PM1, 5′-(T)₂₅-CTGATGGCTCGAGCGCTCTAGAACT-3′ (SEQ ID NO:5). The two underlined sequences are complementary to the first binding members of RM11-1-3mis18 (19 bp) and R11-1-3mis18 (17 bp), respectively. Type 2 oligonucleotides were designed for positive controls, i.e., HjT-Pco3, 5′-(T)₂₅-CTCCCAAATCCTGGCAGGTCAGGCA-3′ (SEQ ID NO:6) and HjT-Pco4, 5′-(T)₂₅-GGCAGGTCAGGCAGTTGCGTTCAAC-3′ (SEQ ID NO:7). Both sequences correspond to two regions of BTCD18 gene (1129-1153 and 1141-1165), respectively). A type 3 oligonucleotide was designed for a negative control, i.e., HjT-Nco1, 5′-(T)₂₅-CTAGTTATTGCTCAGCGG-3′ (SEQ ID NO:8), not homologous to any BTCD18 gene sequence.

The oligonucleotide was dissolved in a probe solution (DR. Probsol, DR.Chip Biotechnology Inc., Taiwan) with a final concentration of 10 μM, spotted, and immobilized on a solid substrate.

The amplification product from each discriminating amplification reaction mixture was diluted with a hybridization buffer in a ratio of 1:(50-100). The diluted fraction was boiled for 5 min, chilled on ice, and applied to the just described solid support. The hybridization reaction was performed at 50-55° C. for 1-2 hours using an oven. Then the solid support was washed with a wash buffer (0.5 mL) (DR. Wash, DR.Chip Biotechnology Inc., Taiwan) for at least three times. Biotin-specific calorimetric detection was performed by incubating the solid substrate with a Blocking Reagent (Roche), which contained alkaline phosphatase-conjugated streptavidin (Promega). Subsequently, the solid substrate was washed three times with the wash buffer, and incubated with NBT/BCIP solution (Roche), which was diluted with a detection buffer in a ratio recommended by the supplier for about 10 min in the dark. The results show that each discriminating amplification product was specifically recognized by its corresponding type 1 oligonucleotide, and all amplification products were recognized by each type 2 oligonucleotide. Detection of colored spots on the positions of HjT-P1 indicated the presence of an amplification product of a mutant allele from the primers HjT-F8a and RM11-1-3mis18. An amplification product of a wild-type allele from the primers HjT-F8a and R11-1-3mis18 was detected as colored spots on the positions of HjT-PM1. Unexpectedly, the detection on the solid substrate was about 10-100 times more sensitive than analysis on ethidium bromide-stained agarose gel.

### EXAMPLE 2

### Detection of a Single Nucleotide Polymorphism Using Genomic DNA Isolated from Blood and Milk Samples

Genomic DNA isolated from blood samples. Two whole blood samples, one from a healthy cow and the other from a BLAD carrier, were obtained from Hsinchu Branch, Taiwan Livestock Research Institute (Council of Agriculture, Executive Yuan). Each whole blood sample was transferred to a tube containing EDTA (1-2 mg/mL) to avoid clotting. To prepare the genomic DNA, the whole blood sample was centrifuged at 3,000×g for 5 min. After centrifugation, three layers were distinguishable: the upper layer was plasma, the intermediate layer contained concentrated leukocytes, and the bottom layer contained concentrated erythrocytes. The genomic DNA was extracted from the intermediate layer using QIAmp Blood Kit (QIAGEN, Hilden, Germany). About 10-20 μg genomic DNA was obtained from 1 mL whole blood sample.

Genomic DNA isolated from milk samples. Two milk samples, one from a healthy cow and the other from a BLAD carrier, were obtained from a local farm. Genomic DNA was extracted from a 15 mL milk sample by the alkalic lysis method as described in Shuster et al. (1992) Proc. Acad. Natl. Sci. USA 89: 9225-9229. The genomic DNA in the aqueous lysate was further purified by adding an organic mixture (phenol/chloroform (1:1)), followed by centrifugation at the maximum speed for 10 min at 4° C. After centrifugation, the aqueous layer was transferred to a tube. The genomic DNA was precipitated with 95% of ice-cold ethanol.

Discrimination amplification. All primers and probes were the same as those described in Example 1. All reagents for amplification reactions were also the same, except two microliters of genomic DNA isolated from blood and milk samples were used for amplification. A three-step amplification reaction was employed with temperature parameters of 95° C. for 4 min; 10 cycles of 95° C. for 60 sec; 52° C. for 60 sec; 72° C. for 60 sec; 25 cycles of 95° C. for 30 sec; 60° C. for 30 sec; 72° C. for 30 sec; and 72° C. for 5 min. The amplification products were analyzed by using the agarose gel electrophoresis and the hybridization method as also described in Example 1. An amplification product of a wild-type allele was detected in the samples isolated from the healthy cow, and amplification products of wild-type and mutant alleles were detected in the samples isolated from the BLAD carrier.

Other Embodiments

All of the features disclosed in this specification may be combined in any combination. Each feature disclosed in this specification may be replace by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, one can change the number and the position of the mismatched base in a discrimination primer to achieve discriminating amplification. Thus, other embodiments are also within the claims.

What is claimed is:

1. A discrimination primer for amplifying a nucleic acid that includes a first base at a position suspected of a polymorphism and a second base immediately 3′ to the first base, the primer comprising:
a first nucleotide, which is located at the 3′ terminus of the primer and contains a base that is complementary to the first base;
a second nucleotide, which is located immediately 5′ to the first nucleotide and contains a base that is not complementary to the second base;
a segment of nucleotides, which is located immediately 5′ to the second nucleotide and is complementary to a part of the nucleic acid that is immediately 3′ to the second base; and
a binding member of a specific binding pair covalently bonded to the 5′ terminus of the segment.

2. The primer of claim 1, wherein the segment is 5 to 50 nucleotides in length.

3. The primer of claim 2, wherein the binding member is an oligonucleotide 6 to 50 nucleotides in length and not complementary to any part of the nucleic acid.

4. The primer of claim 3, wherein the oligonucleotide 10 to 40 nucleotides in length.

5. The primer of claim 2, wherein the binding member is a peptide.

6. The primer of claim 2, wherein the segment is 10 to 40 nucleotides in length.

7. The primer of claim 6, wherein the binding member is an oligonucleotide 6 to 50 nucleotides in length and not complementary to any part of the nucleic acid.

8. The primer of claim 7, wherein the oligonucleotide is 10 to 40 nucleotides in length.

9. The primer of claim 6, wherein the binding member is a peptide.

10. A method for detecting a polymorphism in a nucleic acid, comprising:
providing a nucleic acid including a first base at a position suspected of a polymorphism and a second base immediately 3′ to the first base;
amplifying the nucleic acid with a first primer and a second primer, wherein the first primer includes a first nucleotide, which is located at the 3′ terminus and contains a base that is complementary to the first base, a second nucleotide, which is located immediately 5′ to the first nucleotide and contains a base that is not complementary to the second base; a segment of nucleotides, which is located immediately 5′ to the second nucleotide and is complementary to a part of the nucleic acid that is immediately 3′ to the second base; and a first binding member of a specific binding pair covalently bonded to the 5′ terminus of the segment;
contacting the amplified nucleic acid with a second binding member of the specific binding pair; and
detecting the amplified nucleic acid that binds to the second binding member.

11. The method of claim 10, further comprising amplifying the nucleic acid in the presence of a second first primer, wherein the first nucleotide in one of the two first primers is mutant, and the first nucleotide in the other is wild-type.

12. The method of claim 11, wherein the second primer contains a label at the 5′ terminus.

13. The method of claim 11, wherein the second binding member is immobilized on a solid support.

14. The method of claim 13, wherein the second primer contains a label at the 5′ terminus.

15. The method of claim 13, wherein each of the first binding members of the first primers is, independently, a peptide or an oligonucleotide not complementary to any part of the nucleic acid.

16. The method of claim 15, wherein the first binding members of the first primers are different oligonucleotides.

17. The method of claim 16, wherein the second primer contains a label at the 5′ terminus.

18. The method of claim 10, wherein the second primer contains a label at the 5′ terminus.

19. The method of claim 10, wherein the second binding member is immobilized on a solid support.

20. The method of claim 19, wherein the second primer contains a label at the 5′ terminus.

21. The method of claim 10, wherein the first binding member is an oligonucleotide not complementary to any part of the nucleic acid.

22. The method of claim 10, wherein the first binding member is a peptide.

23. A kit for amplifying a nucleic acid that includes a first base at a position suspected of the polymorphism and a second base immediately 3′ to the first base, the kit comprising a first primer and a second primer, wherein the first primer includes a first nucleotide, which is located at the 3′ terminus and contains a base that is complementary to the first base, a second nucleotide, which is located immediately 5′ to the first nucleotide and contains a base that is not complementary to the second base; a segment of nucleotides, which is located immediately 5′ to the second nucleotide and is complementary to a part of the nucleic acid that is immediately 3′ to the second base; and a binding member of a specific binding pair covalently bonded to the 5′ terminus of the segment.

24. The kit of claim 23, further comprising a second first primer, wherein the first nucleotide in one of the two first primers is mutant, and the first nucleotide in the other is wild-type.

25. The kit of claim 24, wherein the second primer contains a label at the 5′ terminus.

26. The kit of claim 24, wherein each of the first binding members of the first primers is, independently, a peptide or an oligonucleotide not complementary to any part of the nucleic acid.

27. The kit of claim 26, wherein the first binding members of the first primers are a different oligonucleotides.

28. The kit of claim 27, wherein the second primer contains a label at the 5′ terminus.

29. The kit of claim 23, wherein the second primer contains a label at the 5′ terminus.

30. The kit of claim 23, wherein the first binding member is an oligonucleotide not complementary to any part of the nucleic acid.

31. The kit of claim 23, wherein the first binding member is a peptide.

32. The kit of claim 24, wherein each of the first binding members of the first primers is, independently, a peptide or an oligonucleotide that is not complementary to any part of the nucleic acid.

33. The kit of claim 26, wherein the first binding members of the first primers are different oligonucleotides.

34. The kit of claim 27, wherein the second primer includes a label at the 5′ terminus.

35. The kit of claim 23, wherein the second primer includes a label at the 5′ terminus.

36. The kit of claim 23, wherein the first binding member is an oligonucleotide, which is not complementary to any part of the nucleic acid.

37. The kit of claim 23, wherein the first binding member is a peptide.
